(19) United States
(12) Patent Application Publication
(10) Pub. No.: US 2010/0285055 A1
(43) Pub. Date: Nov. 11, 2010

(54) Activation of natural killer (NK) cells and methods of use

(76) Inventors: Sina Bavari, Frederick, MD (US); Kelly L. Warfield, Adamstown, MD (US)

Correspondence Address: U.S. Army Medical Research and Materiel Commnad;Attn: MCMR-JA (Ms. Elizabeth Arwine), 504 Scott Street, Fort Detrick, MD, 21702-5012, US

(21) Appl. No.: 12/661,636

(22) Filed: Mar. 22, 2010

Related U.S. Application Data
(63) Continuation of application No. 11/105,056, filed on Apr. 13, 2005, now abandoned.
(60) Provisional application No. 60/562,803, filed on Apr. 13, 2004.

(51) Int. Cl. A61K 39/12; C07K 14/08; A61K 39/295; A61P 35/00; A61P 37/08; A61P 37/04; A61P 31/14

(52) U.S. Cl. 424/196.11; 424/204.1; 530/350; 424/201.1; 424/202.1

(57) ABSTRACT

The present invention relates to filovirus VLPs and their use in activating innate immunity, specifically natural killer cells, and in enhancing an immune response to an antigen in an animal.

This application is claims the benefit of priority under 35 U.S.C. 119(e) from U.S. Application Ser. No. 60/562,803 filed on Apr. 13, 2004, still pending and herein incorporated by reference in its entirety.

### INTRODUCTION

Marburg (MARV) and Ebola (EBOV) viruses, members of the family Filoviridae, cause an acute and rapidly progressive hemorrhagic fever with mortality rates up to 90% (Feldmann H., 1996, Arch. Virol. Suppl., 11, 77-100). These viruses are fast-acting, with death often occurring within seven to ten days post infection; however, the incubation period is considered to be two to twenty-one days (Borio L., 2002, JAMA, 287, 2391-2405; Peters C. J., 1999, J. Infect. Dis., 179 Suppl. 1, 9-16). Unfortunately, the natural reservoir of filoviruses is not known. Filoviruses are transmitted through contact with bodily fluids or tissues of humans or nonhuman primates (Brown D. W., 1997, Rev. Med. Viral., 7, 239-247; Pinzon J. E., 2004, Am. J. Trop. Med. Hyg., 71, 664-674). Historically, nosocomial transmission often occurs through re-use of incorrectly sterilized needles and syringes, emergency surgical interventions for undiagnosed bleeding when there has been failure to make a correct diagnosis, or while nursing an infected patient through contact with blood, vomit, other infected secretions or infected tissues (Feldmann, 1996, supra). Additionally, filoviruses have also been documented to be transmissible by aerosol (Jaax, N. K., 1995, Lancet, 346, 1669-1671; Johnson E. et al., 1995, Int. J. Exp. Pathol., 76, 227-236; Belanov, 1996, Vopr. Virusol., 41, 32-34). Another disconcerting property of the filoviruses is that they can be fairly stable, even when treated under harsh environmental conditions, and can survive in dried human blood for several days (Belanov, 1996, supra; Frolov, 1996, Vopr. Virusol., 41, 275-277).

The essence of the immune system is built on two separate foundation pillars: one is specific or adaptive immunity characterized by relatively slow response-kinetics and the ability to remember; the other is non-specific or innate immunity exhibiting rapid response-kinetics but lacking memory. The key initiators of innate immunity, including monocytes, macrophages, and dendritic cells (DC), appear to be the primary targets of filovirus infection (Johnson E. et al., 1995, supra; Stroher U. et al., 2001, J. Viral., 75, 11025-11033; Mahanty S. et al., 2003, J. Immunol., 170, 2797-2801; Bosio C. M. et al., 2003, J. Infect. Dis., 188, 1630-1638). EBOV replicates efficiently in DC without eliciting cytokine and chemokine secretion, and infected DC fail to mature and alert other critical mediators of early and adaptive immune responses (Bosio, 2003, supra; Mahanty, 2003, supra). This lack of DC activity most likely results in poor immune responses by natural killer (NK), T, and B cells, which in turn contributes to the uncontrolled spread and growth of the virus. In contrast, the early initiation of innate pro-inflammatory responses correlates with the survival of EBOV-infected humans (Baize S., 1999, Nat. Med., 5, 423-426; Leroy E. M., 2000, Lancet, 355, 2210-2215; Leroy E. M., 2001, Clin. Exp. Immunol., 124, 453-460; Baize S., 2002, Clin. Exp. Immunol., 128, 163-168). Therefore, the rapid initiation of early immune responses may limit EBOV infection, and is critically linked to host survival.

NK cells are key components of the innate immune system, rapidly responding to invading microbes by exocytosis of perforin and granzymes, which mediate the destruction of infected cells (Biron C., 1999, Annu. Rev. Immunol, 17, 189-220). Additionally, NK cell secretion of cytokines such as interferon (IFN)-γ, IFN-α/β, and tumor necrosis factor (TNF)-α serve a dual purpose in that they initiate the immediate activation of anti-microbial pathways in infected cells, followed by modulation of adaptive responses to the pathogen (Biron, 1999, supra; Guidotti L. G., 2001, Annu. Rev. Immunol., 19, 65-91; Lieberman L. A., 2002, 4, 1531-1538). The induction of cytokines and chemokines by viral infections is also known to trigger NK cell activity. Specifically, virus induced IFN-α/β enhances NK cell-mediated cytotoxicity. Alternately, the induction of interleukin (IL)-12 by some viral infections is responsible for the production of high levels of IFN-γ by NK cells, as well as the induction of NK cytotoxic activity (Biron, 1999, supra).

NK cells appear to play a critical role in the immune response to Epstein-Barr virus, murine cytomegalovirus (MCMV), and herpes simplex virus-1 (Scalzo A. A., 2002, Trends Microbiol, 10, 470-474; Rager-Zisman B., 1987, J. Immunol, 138, 884-888; Bukowski J. F., 1985, 161, 40-52). The clinical importance of NK cells to antiviral immunity is documented by the fact that recurrent Herpesvirus infections have been observed in a NK-deficient patient (Biron C. A., 1989, N. Engl. J. Med., 320, 1731-1735). NK cell activity is closely regulated by a myriad of activating and inhibiting cell surface receptors, and consequently, viruses have evolved multiple mechanisms to evade or modulate these receptors. Such mechanisms include the up-regulation of HLA-C and HLA-E molecules on the surface of virus-infected cells, expression of viral MHC homologues to trigger NK inhibitory receptors, and/or the release of cytokine homologues with inhibitory activities (Scalzo, 2002, supra; Biron, 1999, supra; Guidotti, 2001, supra). By contrast, virus-infected cells often down-regulate class I major histocompatibility complex (MHC) on their surface, which then enhances NK cell-mediated lysis due to removal of the inhibitory signals delivered by MHC.

Natural killer cells (NK cells) are also a very early line of defense against tumor cells. They are the cells that are spontaneously cytolytic for certain, but by no means all, tumor lines in culture. NK cells can be characterized by the presence of CD56 and CD16 (human) or NK1.1 or DX5 (mouse) markers and by the absence of the CD3 marker. Because of their non-specific cytotoxic properties for antigen and their efficacy, NK cells constitute a particularly important population of effector cells in the development of immunoadoptive approaches for the treatment of cancer. In this respect, anti-tumoral adoptive immunotherapy approaches have been described in the prior art. NK cells have also been used for experimental treatment of different types of tumors and certain clinical studies have been initiated (Kuppen et al., Int. J. Cancer, 56 (1994) 574; Lister et al., Clin. Cancer Res. 1 (1995) 607; Rosenberg et al., N. Engl. J. Med., 316 (1987) 889). Further, such cells can also be used in vitro for non specific lysis of cells which do not express class I MHC molecules, and more generally any cell which is sensitive to NK cells.

However, adoptive therapy using NK cells (to treat murine or human tumors or other disorders such as infectious diseases) or any other in vitro or in vivo use of such cells involves ex vivo expansion and activation of the NK cells. In this respect, current techniques for activating NK cells are all based on using cytokines, generally in high doses which are not tolerated well by the host. The available data appears to indicate that NK cells do not survive ex vivo and cannot be activated without a nutritive support or without cytokines.

Thus current methods for activating NK cells in vitro involve culturing such cells in the presence of different cytokines (such as IL-1, IL-2, IL-12, IL-15, IFNα, IFNγ, IL-6, IL-4, IL-18 in certain circumstances), used alone or in combination, which activation can be considerably increased by adhesion factors or co-stimulation factors such as ICAM, LFA or CD70. Similarly, in vivo, the efficacy of NK cells in anti-tumoral immunity is not dissociable from co-administration of cytokines such as IL-2/IL-15 or IL-12, IL-18, and IL-10. The activation methodologies described in the prior art thus all depend on using cytokines. Such methods have certain disadvantages, however, linked to the cost of preparing the cytokines, to the toxic nature of many cytokines, which cannot be used in in vivo applications, or to the non-specific nature of many cytokines, the in vivo use of which risks being accompanied by undesirable effects. Further, since the natural killing function is often altered in patients with tumors, the possibility of collecting such cells to activate them ex vivo can be considerably reduced.

There is thus a real need for novel methods for expanding and activating NK cells to enhance both 10, cellular immunity mediated by cytotoxic T lymphocytes and humoral immunity mediated by antibodies. The present application provides a solution to this problem. In particular, the present application demonstrates for the first time the possibility of activating resting NK cells with virus-like particles (VLPs). The present application also describes, for the first time, a method of activating NK cells which is not dependent on the presence of cytokines, and which can thus overcome the disadvantages described in the prior art. The present invention thus describes novel methods for preparing activated natural killer cells and means for carrying out these novel methods.

Therefore, there is a need for compounds which augment the immune response to an immunogen.

### SUMMARY OF THE INVENTION

The present invention satisfies the needs discussed above. The present invention is directed to a composition and method for activating NK cells in order to enhance the immune system response against a foreign cell or organism. When the composition of the invention is administered with an immunogen, the composition enhances the immune response to said immunogen and therefore constitutes a highly effective adjuvant. In addition, we found that Ebola VLPs enhanced the number of natural killer cells in lymphoid tissue. Ebola VLPs containing only the matrix viral protein (VP)40 were sufficient to induce natural killer cells responses and provide protection from infection in the absence of the viral glycoprotein.

We have previously shown that virus-like particles, comprised of the EBOV glycoprotein (GP) and VP40 efficiently mature and activate murine and human myeloid dendritic cells (Warfield K. L., 2003, Proc. Natl. Acad. Sci. USA., 100, 15889-15894; Bosio C. M., 2004, Virology, 326, 280-287). In addition to their potent activation of DC, which are critical mediators of innate and adaptive immune responses, VLP activate T and B cells in vivo following intraperitoneal administration to mice (Warfield, 2003, supra). Therefore, since VLP are highly immunogenic in mice in the absence of adjuvant, we utilized the genome-free Ebola VLPs to study the contribution of NK cells to innate immune responses to lethal EBOV infection. We found that VLPs enhanced the number of natural killer cells in lymphoid tissue. VLPs containing only VP40 were sufficient to induce natural killer cells responses and provide protection from infection in the absence of the viral glycoprotein.

In a first aspect, the invention thus provides a method of activating NK cells that comprises bringing NK cells into contact with Ebola or Marburg VLPs (containing at least VP40 and potentially other viral proteins, including GP, nucleoprotein (NP), VP24, VP30, and/or VP35 of any filovirus subtype or strain). As indicated below, contact between the VLPs and NK cells can be made in vitro, ex vivo, or in vivo. It can comprise either culturing of NK cells in vitro and then exposing the cells in culture to VLPs, or in vivo administration of one or more VLPs.

In a further aspect, the invention concerns the use of VLPs or of a preparation derived from Ebola or Marburg virus VLP-producing cells to activate natural killer cells in vitro, ex vivo or in vivo.

In a further aspect, the invention concerns the use of VLPs or of a preparation derived from VLPs to prepare a composition intended to activate natural killer cells in vivo or enhance proliferation or trafficking of NK cells.

In a yet still further aspect, the present invention concerns a novel population of VLP activated NK cells, and any composition containing them, and uses thereof.

In other aspects, the invention provides a sub-population of NK cells activated by the method of the invention and using these cells to stimulate cytotoxic activity in vivo or in vitro against target cells sensitive to NK cells. In a further aspect, the invention also relates to methods for greatly increasing the cytolytic activity of resting NK cells to produce cytokines including IFN-γ, IL-6, IL-8, and TNF-α.

The invention also concerns novel therapeutic approaches, in particular for treating infectious, tumoral, autoimmune or congenital disorders or for disorders connected to transplantation, for example. In particular, the methods of the invention involve passive transfer (i) of NK cells activated by VLPs ex vivo, or (ii) or a preparation of VLPs to directly activate the NK cells in situ, or (iii) or administration of the VLP in vivo such that they become capable of efficiently activating NK cells, the VLP being administered alone or in association with chemokines or cytokines, used alone or in combination.

In another aspect, the present invention provides a VLP having an adjuvant effect.

### BRIEF DESCRIPTION OF THE DRAWINGS

FIGS. 1A, 1B, 1C and 1D. Ebola virus-like particles (VLPs) induce rapid protective responses against Ebola virus (EBOV) infection. (A) Atomic force micrograph of a VLP (bar=0.25 μm), courtesy of Matt Thompson at Veeco Instruments, Woodbury, New York. (B) C57Bl/6 mice were primed intraperitoneally with 25 μg of VLPs (□) one (n=10), (▴) two (n=10), or (▪) three days (n=30) before challenge, or (◯) irradiated, inactivated (i)EBOV (n=10), or () sucrose-purified supernatants from mock-transfected cells or PBS (n=30) three days before challenge with 100 pfu of mouse-adapted EBOV. Results are plotted as percent survival for each group and the survival curves were constructed using data from two to five separate experiments. Treatment with VLPs one to three days prior to challenge significantly increased the proportion of the mice surviving challenge (P<0.0001) compared to mice treated with iEBOV or sucrose-purified supernatants from mock-transfected cells, based on a one-way Fisher's exact test. (C) One intramuscular injection with (▪) VLPs or (□) PBS was administered to C57Bl/6 mice (n=10/group) three days before challenge with 100 pfu of mouse-adapted EBOV. Results are plotted as percent survival for each treatment group. The data was generated in two separate experiments with five mice per group. A significant increase in survival was observed in VLP-treated mice compared to PBS-treated mice (P<0.0001). (D) One intraperitoneal injection of PBS (unfilled) or VLP (filled) was administered to C57Bl/6 mice three days before challenge with 100 pfu of mouse-adapted EBOV. Serum was collected from the VLP- or PBS-vaccinated mice 4 or 7 days post challenge (dpc) with EBOV and assayed for viral titers by plaque assay. Data are represented as the mean±standard deviation (n=5).

FIGS. 2A, 2B, and 2C. The innate protection against EBOV mediated by VLPs requires functional NK cells. (A) Mediastinal lymph node or splenic cells from mice injected with VLP (filled) or PBS (unfilled) were evaluated for cell surface expression of NK1.1 by flow cytometry. These data represent the average of the number of NK1.1+ cells in each organ +/− standard deviation. The * indicates P≦0.001 for the VLP-injected mice compared to the control mice by student's paired t test (n=5). Similar results were obtained in two separate experiments. (B) NK cell-deficient mice (n=6/group) were injected intraperitoneally with 25 μg of VLPs (▪) or media (□). As controls, C57Bl/6 mice (n=6/group) were administered VLPs (♦) or media (⋄). Three days later the mice were challenged with 100 pfu of mouse-adapted EBOV. Results are plotted as percent survival for each group. A significant decrease in the survival of VLP-treated NK cell-deficient mice was observed, as compared to the VLP-treated C57Bl/6 control mice (P=0.0076). (C) NK cells were depleted from C57Bl/6 mice by intraperitoneal injection of 50 μl of anti-asialoGM antibodies every other day from −5 to +5 days post challenge. Control mice were treated identically using rabbit Ig (Sigma, St. Louis, Mo.). NK cell-depleted mice were injected intraperitoneally with 25 μg of VLPs (▪, n=13) or media (□, n=5) three days before challenge or control-treated mice were administered VLPs (♦, n=15) or media (⋄, n=5) 3 days before challenge. The mice were then challenged with 100 pfu of mouse-adapted EBOV. Percent survival for each group is shown. A significant difference in the survival of VLP-treated NK cell-depleted mice was found, when compared to the VLP-treated C57Bl/6 control mice (P=0.0001).

FIGS. 3A, 3B, 3C, 3D, and 3E. Ebola virus-like particles activate NK cells. (A) NK cells from the livers of unelicited or IL-2-elicited C57Bl/6 mice were incubated overnight with 10 μg of cell-free supernatants from pWRG vector-transfected cells purified on sucrose gradients (designated pWRG and shown by unfilled bar), 100 iU/ml of mouse IL-2 (gray filled bars), or 10 μg of VLP (black filled bars). The supernatants were assayed for IFN-γ by cytometric bead assay. (B and C) NK from the livers of IL-2-elicited C57Bl/6 mice were incubated overnight with media alone, IL-2, or increasing concentrations (0.5-50 μg) of VLPs or inactivated (i)EBOV. The supernatants were assayed for (B) IFN-γ or (C) TNF-α. (D) NK cell preparations stimulated overnight with media or 10 μg of VLPs. The treated NK cells were stained for surface expression of NK1.1 and then fixed, permeabilized, and stained for intracellular IFN-γ. The percent of viable lymphocytes (based on forward and side scatter) which were positive for both NK1.1 and IFN-γ are indicated. The data in this figure represent three experiments of similar design and outcome. (E) NK cells were stimulated with VLPs for (▪) 2 or (▴) 18 hours or (◯) media alone. After the incubation period, the NK cells were added to ⁵¹Cr-labeled YAC-1 cells at varying effector:target ratios, as indicated. The amount of ⁵¹Cr released into the supernatant was determined and the percent specific release calculated. Data are representative of at least two independent experiments.

FIGS. 4A, 4B, 4C and 4D. Ebola virus effects on murine NKs. (A-C) The concentration of IFN-γ (A), MIP-1α (B), or TNF-α (C) in cell supernatants of NK cells exposed to 1 multiplicity of infection (moi) of EBOV-Zaire 95 (◯) or -mouse-adapted (), 10 μg of VLPs (⋄), 100 iU/ml of IL-2 (▴), or media alone (□) was determined over time using ELISA. (D) Viral titers in murine NK cells exposed to Ebola virus. Murine NK cells were infected with 1 moi of EBOV-Zaire 95 (◯) or -mouse-adapted (□). As a control, VeroE6 cells were infected with 1 moi of EBOV-Zaire (▪) or -mouse-adapted (♦). The cell-free supernatants were assayed for growth of EBOV using plaque assay at the indicated times. The data are presented as the number of plaque-forming units (pfu) generated following exposure of one million NK cells over time. These data are representative of three similar and separate experiments.

FIGS. 5A, 5B, 5C, and 5D. Perforin-dependent protection mediated by NK cells against EBOV. (A) NK cells from IL-2-treated C57Bl/6 mice were incubated overnight with 1 (Δ, n=5) or 10 μg/ml (▴, n=20) of VLPs, 50 μg/ml of inactivated EBOV (▪, n=10), 10 μg/ml of polyI:C (♦, n=5), or media alone (◯, n=10). Naive recipient mice were injected with 5×10⁶ treated NK cells and challenged 6 hours later with 10 pfu of mouse-adapted EBOV. The results are presented on Meier-Kaplan survival curves. By a one-way Fisher's exact test, transfer of NK cells treated with 10 μg of VLPs, but not 1 μg of VLPs or 50 μg of inactivated EBOV, significantly increased the proportion of the mice surviving challenge (P<0.0001) compared to mice receiving media-treated NK cells. (B) NK cells were isolated from the livers of IL-2 treated mice by negative selection. These highly-enriched NK cell preparations were then incubated overnight with (▴) VLPs or (◯) media alone. Alternately, the NK cell preparation was depleted of NK1.1⁺ cells using magnetic beads and this NK cell-depleted (>90% reduction) population was stimulated with VLPs (Δ). Following overnight incubation, the cell populations were injected into naïve recipient mice (n=10/group) and the mice were challenged 6 hours later with 10 pfu of mouse-adapted EBOV. The results are presented as percent survival for each group and the survival curves were generated using data from two separate experiments with five mice per group. A significant increase in survival was observed in mice receiving the VLP-treated NK cells when compared mice that received media-treated NK cells (P<0.0001). In contrast, there was not a significant difference in survival between the mice receiving cell preparations depleted of NK cells and treated with VLPs, when compared to mice receiving media-treated NK cells (P=0.5891). (C) NK cells were harvested from IFN-γ-deficient (C57Bl/6 background) mice. The NK cells were incubated overnight with VLPs () or media alone (◯) and then transferred to naïve C57Bl/6 mice. As a control, NK cells from C57Bl/6 mice were incubated overnight with VLPs (▴) and transferred to naïve recipient C57Bl/6 mice. The recipient mice were then challenged with 10 pfu of EBOV and monitored for illness. The results are presented as percent survival for each group (n=10) and the survival curves were generated using data from two separate experiments with five treated mice per group. A significant increase in survival was observed in mice receiving the VLP-treated NK cells isolated from IFN-γ-deficient or wild-type C57Bl/6 mice (P=0.0007 or 0.0015, respectively) when compared to control mice that received media-treated NK cells. (D) NK cells were harvested from perforin-deficient (BALB/c background) mice and were incubated overnight with VLPs (♦) or media alone (⋄). As a control, NK cells from BALB/c mice were incubated overnight with VLPs (▴). Five million stimulated NK cells were then transferred to naïve BALB/c mice by intraperitoneal injection. The recipient mice were then challenged with 10 pfu of EBOV and monitored for illness. The results are presented as percent survival for each group (n=10) and the survival curves were generated using data from two separate experiments with five treated mice per group. A significant increase in survival was observed in mice receiving the VLP-treated NK cells isolated from wild-type BALB/c mice (P=0.0007) when compared to control mice that received media-treated NK cells. However, mice receiving VLP-treated NK cells from perforin-deficient mice did not have a significant increase in survival compared to control mice that received media-treated NK cells (P=0.5000).

FIGS. 6A, 6B, 6C, 6D, and 6E. Ebola virus VP40 is sufficient to induce NK cell responses. (A) Antibodies, including either 30 μg of an irrelevant monoclonal to human (h) CD2, a pool of three monoclonals against GP (αGP), or a monoclonal that recognizes VP40 (αVP40), or 30 μl of sera from mice vaccinated with Venzuelan equine encephalitis replicon particles expressing GP (VRP-VP40) or Lassa virus GP (VRP-Lassa), were pre-incubated for 1 hour on ice with 10 μg of VLPs. Purified NK cells were then incubated overnight with the antibody-VLP complexes and the concentration of IFN-γ in the NK cell supernatants was determined. The data are shown as percent of the control sample (range: 510-829 μg/ml), which was calculated by the equation: [IFN-γ secretion with test antibody/IFN-γ secretion with control antibody (hCD2)]×100%. The graph shows the mean of three experiments with errors bars demonstrating the standard deviation from the mean of the three experiments. The * indicates a significant inhibition (P<0.05) compared to the control culture as determined by paired student's t test. (B) VLPs, made of GP and VP40, or VLP_{VP40}, containing VP40 alone, were incubated with NK cells overnight and then the levels of IFN-γ in the NK cell supernatants were determined. Data are representative of four independent experiments. (C) NK cells were incubated with VLPs (▴), VLP_{VP40} (), or media alone (◯) overnight and added to ⁵¹Cr-labeled YAC-1 cells at varying effector:target ratios. The amount of ⁵¹Cr released into the supernatant was determined and the percentage specific release determined. Similar results were obtained in two separate experiments. (D) NK cells were incubated overnight with 10 μg/ml of VLPs (▴), VLP_{VP40} (), or media alone (◯). Naïve mice were injected intraperitoneally with five million of the VLP- or media-treated NK cells and then challenged 6 hours later with 10 pfu of mouse-adapted Ebola virus. The results are presented as percent survival for each group (n=10) and the survival curves were generated using data from two separate experiments with five treated mice per group. A significant increase in survival was observed in mice receiving the VLP- or VLP_{VP40}-treated NK cells (P=0.0027 or 0.0001, respectively) when compared to control mice that received media-treated NK cells. (E) C57Bl/6 mice were primed with 10 μg of VLPs (▴), VLP_{VP40} (), or PBS (◯) three days before challenge with 10 pfu of mouse-adapted EBOV. The data are presented as percent survival for each group (n=10) and the survival curves were generated using data from two separate experiments with five treated mice per group. A significant increase in the proportion of mice surviving was observed in mice treated with VLPs (P<0.0001) or VLP_{VP40} (P<0.0001) when compared to control mice injected with PBS.

### DETAILED DESCRIPTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are hereinafter described.

Filoviruses. The filoviruses (e.g. Ebola virus (EBOV) and Marburg virus (MBGV)) cause acute hemorrhagic fever characterized by high mortality. Humans can contract filoviruses by infection in endemic regions, by contact with imported primates, and by performing scientific research with the virus. However, there currently are no available vaccines or effective therapeutic treatments for filovirus infection. The virions of filoviruses contain seven proteins which include a surface glycoprotein (GP), a nucleoprotein (NP), an RNA-dependent RNA polymerase (L), and four virion structural proteins (VP24, VP30, VP35, and VP40).

Subject. Includes human, animal, avian, e.g., horse, donkey, pig, mouse, hamster, monkey, chicken, and insect such as mosquito.

Virus-like particles (VLP). This refers to a structure which resembles the outer envelope of the native virus antigenically and morphologically. The virus-like particles are formed in vitro upon expression, in a cell, of viral surface glycoprotein (GP) and a virion structural protein, VP40. It may be possible to produce VLPs by expressing only portions of GP and VP40.

Animal: As used herein, the term “animal” is meant to include, for example, humans, sheep, horses, cattle, pigs, dogs, cats, rats, mice, birds, reptiles, fish, insects and arachnids

A “microbial antigen” as used herein is an antigen of a microorganism and includes, but is not limited to, infectious virus, infectious bacteria, parasites and infectious fungi. Such antigens include the intact microorganism as well as natural isolates and fragments or derivatives thereof and also synthetic or recombinant compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to the skilled artisan.

Antigenic determinant: As used herein, the term “antigenic determinant” is meant to refer to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

Adjuvants are compounds which enhance the immune systems response when administered with antigen producing higher antibody titer and prolonged host response. Commonly used adjuvants include incomplete Freund's adjuvant, which consists of a water in oil emulsion, Freund's Complete adjuvant, which comprises the above with the addition of Mycobacterium tuberculosis, Montanide, and alum. The difficulty, however, in using these materials in humans, for example, is that they are toxic or may cause the host to develop lesions at the site of injection. In addition, these adjuvants fail to act as immunopotentiating agents when administered orally or enterally.

Bound: As used herein, the term “bound” refers to binding that may be covalent, e.g., by chemically coupling to a virus-like particle, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like. The term also includes the enclosement, or partial enclosement, of a substance. The term “bound” is broader than and includes terms such as “coupled,” “fused,” “enclosed” and “attached.” Moreover, with respect to the antigen being bound to the virus-like particle the term “bound” also includes the enclosement, or partial enclosement, of the antigen. Therefore, with respect to the antigen being bound to the virus-like particle the term “bound” is broader than and includes terms such as “coupled,” “fused,” “enclosed”, “packaged” and “attached.” For example, the antigen can be enclosed by the VLP without the existence of an actual binding, neither covalently nor non-covalently, such that the antigen is held in place by mere “packaging.”

Coupled: As used herein, the term “coupled” refers to attachment by covalent bonds or by strong non-covalent interactions, typically and preferably to attachment by covalent bonds. Any method normally used by those skilled in the art for the coupling of biologically active materials can be used in the present invention.

Fusion: As used herein, the term “fusion” refers to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

Epitope: As used herein, the term “epitope” refers to continuous or discontinuous portions of a polypeptide having antigenic or immunogenic activity in an animal, preferably a mammal, and most preferably in a human. An epitope is recognized by an antibody or a T cell through its T cell receptor in the context of an MHC molecule. An “immunogenic epitope,” as used herein, is defined as a portion of a polypeptide that elicits an antibody response or induces a T-cell response in an animal, as determined by any method known in the art. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art. Immunospecific binding excludes non-specific binding but does not necessarily exclude cross-reactivity with other antigens. Antigenic epitopes need not necessarily be immunogenic. Antigenic epitopes can also be T-cell epitopes, in which case they can be bound immunospecifically by a T-cell receptor within the context of an MHC molecule. An epitope can comprise 3 amino acids in a spatial conformation which is unique to the epitope. Generally, an epitope consists of at least about 5 such amino acids, and more usually, consists of at least about 8-10 such amino acids. If the epitope is an organic molecule, it may be as small as Nitrophenyl.

Immune response: As used herein, the term “immune response” refers to a humoral immune response and/or cellular immune response leading to the activation or proliferation of B- and/or T-lymphocytes and/or antigen presenting cells. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. An “immunogenic polypeptide” is a polypeptide that elicits a cellular and/or humoral immune response, whether alone or linked to a carrier in the presence or absence of an adjuvant. Preferably, the antigen presenting cell may be activated.

Immunization: As used herein, the terms “immunize” or “immunization” or related terms refer to conferring the ability to mount a substantial immune response (comprising antibodies and/or cellular immunity such as effector CTL) against a target antigen or epitope. These terms do not require that complete immunity be created, but rather that an immune response be, produced which is substantially greater than baseline. For example, a mammal may be considered to be immunized against a target antigen if the cellular and/or humoral immune response to the target antigen occurs following the application of methods of the invention.

Mixed: As used herein, the term “mixed” refers to the combination of two or more substances, ingredients, or elements that are added together, are not chemically combined with each other and are capable of being separated.

Packaged: The term “packaged” as used herein refers to the state of an antigen, in particular a peptide or nucleic acid in relation to the VLP. The term “packaged” as used herein includes binding that may be covalent, e.g., by chemically coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like. The term “packaged” includes terms such as “coupled” and “attached”, and in particular, and preferably, the term “packaged” also includes the enclosement, or partial enclosement, of a substance. For example, the antigen can be enclosed by the VLP without the existence of an actual binding, neither covalently nor non-covalently.

Polypeptide: As used herein, the term “polypeptide” refers to a molecule composed of monomers (amino acids) linearly linked by amide bonds (also known as peptide bonds). It indicates a molecular chain of amino acids and does not refer to a specific length of the product. Thus, peptides, oligopeptides and proteins are included within the definition of polypeptide. This term is also intended to refer to post-expression modifications of the polypeptide, for example, glycosylations, acetylations, phosphorylations, and the like. A recombinant or derived polypeptide is not necessarily translated from a designated nucleic acid sequence. It may also be generated in any manner, including chemical synthesis.

Effective Amount: As used herein, the term “effective amount” refers to an amount necessary or sufficient to realize a desired biologic effect. An effective amount of the composition would be the amount that achieves this selected result, and such an amount could be determined as a matter of routine by a person skilled in the art. For example, an effective amount for treating an immune system deficiency could be that amount necessary to cause activation of the immune system, resulting in the development of an antigen specific immune response upon exposure to antigen. The term is also synonymous with “sufficient amount.” The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

Treatment: As used herein, the terms “treatment”, “treat”, “treated” or “treating” refer to prophylaxis and/or therapy. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse.

Vaccine: As used herein, the term “vaccine” refers to a formulation which contains the composition of the present invention and which is in a form that is capable of being administered to an animal. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses. Optionally, the vaccine of the present invention additionally includes an adjuvant which can be present in either a minor or major proportion relative to the compound of the present invention. The term “adjuvant” as used herein refers to non-specific stimulators of the immune response or substances that allow generation of a depot in the host which when combined with the vaccine of the present invention provide for an even more enhanced immune response.

A variety of adjuvants can be used. Examples include incomplete Freund's adjuvant, aluminum hydroxide and modified muramyldipeptide.

As indicated above, a first aspect of the invention thus concerns a method for activating NK cells using VLPs. This method comprises bringing NK cells into the presence of VLPs or a preparation derived from VLPs. The present invention is based on a demonstration by the Applicant of the capacity of VLPs to activate resting NK cells.

Filovirus VLPs and their production were described elsewhere (U.S. patent application Ser. No. 10/289,839 filed on Nov. 7, 2002, herein incorporated by reference in its entirety). Briefly, the method includes expressing viral glycoprotein GP and the virion structural protein, VP40 in cells in vitro, ex vivo, or in vivo by administration of DNA fragments which encode these proteins into the desired cells.

Therefore, DNA fragments which encode any of the Ebola Zaire 1976 or 1995 (Mayinga isolate) GP and VP40 proteins (Accession # AY142960 contains the whole genome of Ebola Zaire, with individual genes including GP and VP40 specified in this entry, VP40 gene nucleotides 4479-5459, GP gene 6039-8068) are inserted into a mammalian expression vector, specifically, pWRG7077, and transfected into cells. The entire Marburg (Musoke subtype) genome has been deposited in accession # NC_{—}001608 for the entire genome, with individual genes specified in the entry, VP40 gene 4567-5478, GP gene 5940-7985, NP gene 103-2190. The protein ID for Ebola VP40 is AAN37506.1, for Ebola GP is AAN37507.1, for Marburg VP40 is CAA78116.1, and for Marburg GP is CAA78117.1.

The vector can take the form of a plasmid, a eukaryotic expression vector such as pcDNA3.1, pRcCMV2, pZeoSV2, or pCDM8, which are available from Invitrogen, or a virus vector such as baculovirus vectors, retrovirus vectors or adenovirus vectors, alphavirus vectors, and others known in the art. The minimum requirement is a promoter that is functional in mammalian cells for expressing the gene.

A suitable construct for use in the method of the present invention is pWRG7077 (4326 bp) (PowderJect Vaccines, Inc., Madison, Wis.). pWRG7077 includes a human cytomegalovirus (hCMV) immediate early promoter and a bovine growth hormone polyA addition site. Between the promoter and the polyA addition site is Intron A, a sequence that naturally occurs in conjunction with the hCMV IE promoter that has been demonstrated to increase transcription when present on an expression plasmid. Downstream from Intron A, and between Intron A and the polyA addition sequence, are unique cloning sites into which the desired DNA can be cloned. Also provided on pWRG7077 is a gene that confers bacterial host-cell resistance to kanamycin. Any of the fragments that encode Ebola GP, Ebola VP40, Marburg GP, and Marburg VP40 can be cloned into one of the cloning sites in pWRG7077, using methods known to the art.

All filoviruses have GP proteins that have similar structure, but with allelic variation. By allelic variation is meant a natural or synthetic change in one or more amino acids which occurs between different serotypes or strains of Ebola or Marburg virus and does not affect the antigenic properties of the protein. There are different strains of Ebola (Zaire 1976, Zaire 1995, Reston, Sudan, and Ivory Coast with 1-6 species under each strain). Marburg has species Musoke, Ravn, Ozolin, Popp, Ratayczak, Voege that have 78% homology among these different strains. It is reasonable to expect that similar VLPs from other filoviruses can be prepared by using the concept of the present invention described for MBGV and EBOV, i.e. expression of GP and VP40 genes from other filovirus strains or subtypes would result in VLPs specific for those strains.

Host cells were stably transformed or transfected with the above-described recombinant DNA constructs or expressing said DNA. The host cell can be prokaryotic (for example, bacterial), lower eukaryotic (for example, yeast or insect) or higher eukaryotic (for example, all mammals, including but not limited to mouse and human). Both prokaryotic and eukaryotic host cells may be used for expression of the desired coding sequences when appropriate control sequences which are compatible with the designated host are used. Host cells include all cells susceptible to infection by filovirus.

Among prokaryotic hosts, E. coli is the most frequently used host cell for expression. General control sequences for prokaryotes include promoters and ribosome binding sites. Transfer vectors compatible with prokaryotic hosts are commonly derived from a plasmid containing genes conferring ampicillin and tetracycline resistance (for example, pBR322) or from the various pUC vectors, which also contain sequences conferring antibiotic resistance. These antibiotic resistance genes may be used to obtain successful transformants by selection on medium containing the appropriate antibiotics. Please see e.g., Maniatis, Fitsch and Sambrook, Molecular Cloning; A Laboratory Manual (1982) or DNA Cloning, Volumes I and II (D. N. Glover ed. 1985) for general cloning methods.

In addition, the filovirus gene products can also be expressed in eukaryotic host cells such as yeast cells and mammalian cells. Saccharomyces cerevisiae, Saccharomyces carlsbergensis, and Pichia pastoris are the most commonly used yeast hosts. Control sequences for yeast vectors are known in the art. Mammalian cell lines available as hosts for expression of cloned genes are known in the art and include many immortalized cell lines available from the American Type Culture Collection (ATCC), such as HEPG-2, CHO cells, Vero cells, baby hamster kidney (BHK) cells and COS cells, to name a few. Suitable promoters are also known in the art and include viral promoters such as that from SV40, Rous sarcoma virus (RSV), adenovirus (ADV), bovine papilloma virus (BPV), and cytomegalovirus (CMV). Mammalian cells may also require terminator sequences, poly A addition sequences, enhancer sequences which increase expression, or sequences which cause amplification of the gene. These sequences are known in the art.

The transformed or transfected host cells can be used as a source of DNA sequences described above. When the recombinant molecule takes the form of an expression system, the transformed or transfected cells can be used as a source of the VLP described below.

Cells may be transfected with one or more expression vector expressing filovirus GP and VP40 using any method known in the art, for example, calcium phosphate transfection as described in the examples. Any other method of introducing the DNA such that the encoded proteins are properly expressed can be used, such, as viral infection, electroporation, to name a few.

For preparation of VLPs, supernatants are collected from the above-described transfected cells, preferably 60 hours post-transfection. Other times can be used depending on the desired number of intact VLPs. Our endpoint is the greatest number of intact VLPs, we could use other times which will depend on how we express the genes. Presumably an inducible system would not require the same length of incubation as transient transfections. The supernatants will undergo a low speed spin to reduce contamination from cellular material and then be concentrated by a high speed spin. The partially purified material is then separated on a 10-60% sucrose gradient. The isolation technique will depend upon factors such as the specific host cells used, concentration, whether VLPs remains intracellular or are secreted, among other factors. The isolated VLPs are about 95% pure with a low enough endotoxin content for use as a vaccine. In these instances, the VLP used will preferably be at least 10-30% by weight, more preferably 50% by weight, and most preferably at least 70-90% by weight. Methods of determining VLP purity are well known and include SDS-PAGE densitometric methods.

The resulting VLPs are not homogeneous in size and exhibit conformational, neutralizing epitopes found on the surface of authentic Ebola or Marburg virions. The VLPs are comprised of GP and VP40. Other proteins can be added such as NP, VP24, VP30, and VP35 without affecting the structure or decreasing the efficiency of VLP production (Kallstrom et al., 2005, J. Virol. Methods, in press).

While these results are novel and unexpected, based on the teachings of this application, one skilled in the art may achieve greater VLP yields by varying conditions of transfection and separation.

The results presented in the present application demonstrate that resting NK cells, co-cultivated in the presence of VLPs, are very strongly activated for their lytic capacity and for the production of IFNγ and other cytokines. Further, the activated cells obtained lyse NK cell-sensitive targets, as well as virus-infected cells. These results thus demonstrate that VLPs or preparations derived from VLPs have the capacity to induce activation of NK cells in vitro, ex vivo, and to enhance proliferation, trafficking and activation of NK cells in vivo. This activation can stimulate in vitro lysis of NK sensitive cells and in vivo natural immunity of a host organism, and can thus lead to in vivo elimination of tumors, infected cells, or can be involved in other pathological processes (autoimmune diseases, graft rejection, graft versus host disease, etc. . . . ), and can be used as an adjuvant.

More particularly, the term “activation” of'NK cells within the context of the invention designates an increase in the production of IFNγ, TNFα, IL-6, IL-8 and/or the cytotoxic activity of NK cells. These parameters can easily be measured using techniques which are known to the skilled person and are illustrated in the examples. In addition, this activation may be due to a significant increase in the survival of NK cells in vitro. More particularly, the NK cell activation within the context of the invention is independent of the use of conventional cytokines. The term “activated” NK cells as used within the context of the invention designates NK cells with at least one of the properties mentioned above or may also be measured by the upregulation of cell surface markers.

The NK cell activation method of the invention can be carried out in vitro, ex vivo or directly in vivo.

For effective in vitro or ex vivo activation, certain parameters should advantageously be satisfied such as the ratio of NK cells to VLPs and/or the co-incubation time. Thus, the experiments carried out by the Applicants have demonstrated that the best performances of the in vitro or ex vivo activation method were obtained when the initial NK cell to VLP ratio was in the range 0.01 to 100 g per million NK cells, preferably in the range 0.05 to 50 g per million NK cells. It should be understood that the skilled person is free to adapt this ratio depending on the cell population used, taking into account the stifling effect of NK cells which can be observed when the quantity of VLPs is too high, and the low level of activation which can be observed when the number of VLPs is too low. The time of exposure can also be adapted by the skilled person as a function of the cell populations used. In general, optimal NK cell activation is observed after VLP exposure for a period in the range about 6 to 48 hours. The exposure periods indicated above can in particular produce the best combination between the proportion of activated NK cells and the proportion of viable cells. It should be noted in this respect that, during VLP activation, NK cell proliferation is observed (a factor of about 2). Because of this, the method of the invention can produce activated NK cells without the need to use cytokines, and with improved yields.

NK cells can be obtained for the present invention using different techniques which are known to the skilled person. More particularly, these cells can be obtained by different isolation and enrichment methods using peripheral blood mononuclear cells (lymphoprep, leucapheresis, etc.). Thus these cells can be prepared by Percoll density gradients (Timonen et al., J. Immunol. Methods 51 (1982) 269), by negative depletion methods (Zarling et al., J. Immunol. 127 (1981) 2575) or by FACS sorting methods (Lanier et al., J. Immunol. 131 (1983) 1789). These cells can also be isolated by column immunoadsorption using an avidin-biotin system (Handgretinger et al., J. Clin. Lab. Anal. 8 (1994) 443) or by immunoselection using microbeads grafted with antibodies (Geiselhart et al., Nat. Immun. 15 (1996-97) 227). It is also possible to use combinations of these different techniques, optionally combined with plastic adherence methods.

These different techniques can produce cell populations which are highly enriched in resting NK cells, preferably comprising more than 70% of resting NK cells. More preferably, the NK cell populations used to carry out the invention generally comprise more than 30% of NK cells, advantageously more than 50%. The purity of the cell populations can be improved if necessary using specific antibodies for positive selection such as anti-CD56 antibodies and/or anti-CD16 antibodies (for humans) or anti-NK1.1 or anti-DX5 antibodies and/or anti-CD3, -CD4, CD8, CD14, CD19, or -CD20 antibodies for depletion of the unwanted cell populations. The NK cells can be preserved in a culture medium in a frozen form for subsequent use. Advantageously, the NK cells are prepared extemporaneously, i.e., they are used for activation after production.

NK cell activation in vitro can be carried gut in any suitable cell culture apparatus, preferably under sterile conditions. In particular, they may be plates, culture dishes, flasks, pouches, etc. Exposure to VLPs is carried out in any medium suitable for VLPs and NK cells. More generally, it may be a commercially available culture medium for culturing mammalian cells, preferably, RPMI-1640 media.

In a typical experiment, the activated character of the NK cells is monitored by measuring the IFNγ production in the supernatant and measuring the cytotoxicity against target cells. The NK cells are also counted (for example using trypan blue) and analysed (for example by flow cytometry) for expression of characteristic markers (such as NK1.1 or DX5 in the mouse or CD16 and CD56 in humans or nonhuman primates) and to evaluate the cell mortality.

When the NK cells have been activated in this manner, the NK cells can be separated from the VLPs, or the NK cell:VLP mixture can be harvested directly. In this respect, the invention also provides a composition comprising NK cells and VLPs. As indicated above, they are advantageously activated NK cells. Finally, in these compositions of the invention, the cell populations are preferably autologous, i.e., from the same organism. Preferred compositions of the invention generally comprise at least 10%, preferably 20% to 60%, more preferably 30% to 60% of NK cells. The invention' also concerns any composition comprising activated NK cells as described in the present application. The compositions of the invention can be packaged in any suitable apparatus such as pouches, flasks, ampules, syringes, vials, etc., and can be (cold) stored or used extemporaneously, as described below. Advantageously, these compositions comprise 10⁴ to 10⁹ NK cells, preferably about 10⁶ to 10⁹ (in particular for administration to humans) or 10⁵ to 10⁷ (in particular for administration to mice).

In a further implementation, the method of the invention comprises in vitro, ex vivo or in vivo activation of NK cells by bringing NK cells into the presence of a preparation of VLPs. The preparation derived from VLPs can be any preparation or membranous fraction of VLPs, a lysate of VLPs, or the purified VP40 in its entirety or an immunogenic portion of VP40.

As illustrated in the present application, the NK cells can be activated not only in the presence of VLPs, but also in the presence of membrane preparations thereof or in the presence of VP40.

In a further variation, the method of the invention comprises in vitro, ex vivo or in vivo NK cell activation by bringing the NK cells into the presence of VLP, membranous fractions of VLPs, or isolated, purified, VP40.

The results shown in the present application illustrate the specific nature of the activation of NK cells by VLPs, and thus indicate the involvement of VP40 in carrying out this effect. Therefore VP40, or any preparation containing it, or any derivative or recombinant forms of this factor and the corresponding nucleic acids, can thus also be used in vitro or in vivo to activate NK cells, in particular for anti-tumoral or anti-viral immunization applications. Further, the term “derived” also indicates that the compositions of the invention can comprise any variant or recombinant form of the VLPs or VP40 identified above.

In a further implementation of the invention, the method of the invention comprises in vivo activation of NK cells by providing VLPs in vivo. This in vivo exposure to VLPs can exert an in situ activation of NK cells and can thus reinforce the natural immunity of an organism, in particular against tumour or infected cells.

Administration can be carried out by injection, for example, preferably by subcutaneous or systemic injection of VLPs or polynucleotides encoding VP40 and GP which upon expression in a cell will produce VLPs, or a polynucleotide encoding VP40 or a derivative or variant thereof. Injection is preferably a local or regional injection, in particular into the site or close to the site to be treated, in particular close to a tumor. Injections are generally carried out on the basis of cell doses of 0.01 to 1 mg of VLPs or more per 10⁶ NK cells. Further, the skilled person can adapt the injection protocol to the situation (preventative, curative, isolated tumors, metastases, extended or local infection, etc.). Thus it is possible to provide VLPs in a passive transfer by repeated administration, for example 1 or 2 administrations per week, over several months.

In accordance with the present invention, there is also provided a method for enhancing an immune response to an antigen in a human or an animal which comprises administering to said human or animal an immune composition comprising VLP and at least one antigen, wherein, said antigen can be part of the VLP itself, or administered concomitantly with the antigen but not directly linked to said VLP. The antigen can be a peptide, nucleic acid, can be coupled to, fused to, or otherwise attached to or enclosed by, i.e. bound to, or packaged in the VLP.

A substance which “enhances” an immune response refers to a substance in which an immune response is observed that is greater or intensified or deviated in any way with the addition of the substance when compared to the same immune response measured without the addition of the substance. For example, the lytic activity of cytotoxic T cells can be measured, e.g. using a ⁵¹Cr release assay, with and without the substance. The amount of the substance at which the CTL lytic activity is enhanced as compared to the CTL lytic activity without the substance is said to be an amount sufficient to enhance the immune response of the animal to the antigen. In a preferred embodiment, the immune response in enhanced by a factor of at least about 2, more preferably by a factor of about 3 or more. The amount or type of cytokines secreted may also be altered. Alternatively, the amount of antibodies induced or their subclasses may be altered

In yet another embodiment, the antigen or antigen mixture can be selected from the group consisting of: (1) a polypeptide or organic molecule suited to induce an immune response against cancer cells; (2) a polypeptide or organic molecule suited to induce an immune response against an infectious disease; (3) a polypeptide or organic molecule suited to induce an immune response against allergens; (4) a polypeptide or organic molecule suited to induce an improved response against self-antigens; (5) a polypeptide or organic molecule suited to induce an immune response in farm animals or pets; and (6) an organic molecule suited to induce a response against a drug, a hormone or a toxic compound.

Examples of infectious viruses that have been found in humans include but are not limited to: Retroviridae (e.g. human immunodeficiency viruses, such as HIV-1 (also referred to as HTLV-III, LAV or HTLV-III/LAV, or HIV-III); and other isolates, such as HIV-LP); Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals. Such gram positive bacteria include, but are not limited to, Pasteurella species, Staphylococci species and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps. (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, Actinomyces israelli and Chlamydia.

Examples of infectious fungi include: Cryptococcus neoformans, Histoplasma capsulatum, Coccidioides immitis, Blastomyces dermatitidis, Chlamydia trachomatis and Candida albicans. Other infectious organisms (i.e., protists) include: Plasmodium such as Plasmodium falciparum, Plasmodium malariae, Plasmodium ovale, Plasmodium vivax, Toxoplasma gondii and Shistosoma.

Other medically relevant microorganisms have been descried extensively in the literature, e.g., see C. G. A. Thomas, “Medical Microbiology”, Bailliere Tindall, Great Britain 1983, the entire contents of which is hereby incorporated by reference.

The compositions and methods of the invention are also useful for treating cancer by stimulating an antigen-specific immune response against a cancer antigen. A “tumor antigen” as used herein is a compound, such as a peptide, associated with a tumor or cancer and which is capable of provoking an immune response. In particular, the compound is capable of provoking an immune response when presented in the context of an MHC molecule. Tumor antigens can be prepared from cancer cells either by preparing crude extracts of cancer cells, for example, as described in Cohen, et al., Cancer Research, 54:1055 (1994), by partially purifying the antigens, by recombinant technology or by de novo synthesis of known antigens. Tumor antigens include antigens that are antigenic portions of or are a whole tumor or cancer polypeptide. Such antigens can be isolated or prepared recombinantly or by any other means known in the art. Cancers or tumors include, but are not limited to, biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas.

Allergens also serve as antigens in vertebrate animals. The term “allergen”, as used herein, also encompasses “allergen extracts” and “allergenic epitopes.” Examples of allergens include, but are not limited to: pollens (e.g. grass, ragweed, birch and mountain cedar); house dust and dust mites; mammalian epidermal allergens and animal danders; mold and fungus; insect bodies and insect venom; feathers; food; and drugs (e.g. penicillin). See Shough, H. et al., REMINGTON'S PHARMACEUTICAL SCIENCES, 19th edition, (Chap. 82), Mack Publishing Company, Mack Publishing Group, Easton, Pa. (1995), the entire contents of which is hereby incorporated by reference. Thus, immunization of individuals with allergens mixed with virus like particles should be beneficial not only before but also after the onset of allergies.

The compositions of the invention can be combined, optionally, with a pharmaceutically-acceptable carrier. The term “pharmaceutically-acceptable carrier” as used herein means one or more compatible solid or liquid fillers, diluents or encapsulating substances which are suitable for administration into a human or other animal. The term “carrier” denotes an organic or inorganic ingredient, natural or synthetic, with which the active ingredient is combined to facilitate the application.

In yet another embodiment of the invention, the composition is introduced into an animal subcutaneously, intramuscularly, intranasally, intradermally, intravenously or directly into a lymph node. In an equally preferred embodiment, the immune enhancing composition, whether VLP alone or VLP with a desired antigen, is applied locally, near a tumor or local viral reservoir against which one would like to vaccinate.

The present invention also relates to a vaccine comprising an immunologically effective amount of the immune enhancing composition of the present invention together with a pharmaceutically acceptable diluent, carrier or excipient. In a preferred embodiment, the vaccine further comprises at least one adjuvant, such as Alum or incomplete Freund's adjuvant. The invention also provides a method of immunizing and/or treating an animal comprising administering to the animal an immunologically effective amount of the disclosed vaccine.

The route of injection is preferably subcutaneous or intramuscular, but it would also be possible to apply the CpG-containing VLPs intradermally, intranasally, intravenously or directly into the lymph node. In an equally preferred embodiment, the CpG-containing VLPs mixed with antigen are applied locally, near a tumor or local viral reservoir against which one would like to vaccinate.

The vaccine may comprise two or more antigens depending on the desired immune response. The antigens may also be modified so as to further enhance the immune response. Preferably, proteins or peptides derived from viral or bacterial pathogens, from fungi or parasites, as well as tumor antigens (cancer vaccines) or antigens with a putative role in autoimmune disease are used as antigens (including derivatized antigens like glycosylated, lapidated, glycolipidated or hydroxylated antigens). Furthermore, carbohydrates, lipids or glycolipids may be used as antigens themselves. The derivatization process may include the purification of a specific protein or peptide from the pathogen, the inactivation of the pathogen as well as the proteolytic or chemical derivatization or stabilization of such a protein or peptide. Alternatively, also the pathogen itself may be used as an antigen. The antigens are preferably peptides or proteins, carbohydrates, lipids, glycolipids, or mixtures thereof.

According to a preferred embodiment, T cell epitopes are used as antigens. Alternatively, a combination of T cell epitopes and B cell epitopes may also be preferred.

The VLP described herein can be used alone as an immunopotentiator or adjuvant to enhance an immune response in humans or animals against targeted antigens. It is preferable that the VLP be administered concomitantly with the antigen against which an immune response must be raised. However, the adjuvant VLP can be administered previously or subsequently to, depending on the needs, the administration of the antigen to humans or animals.

The present invention will be more readily understood by referring to the following examples which are given to illustrate the invention rather than to limit its scope.

The following Materials and Methods were used in the Examples below.

Virus and cell lines. The wild-type strain of EBOV-Zaire was originally isolated from a fatally-infected human in 1995 (Jahrling et al., 1996, Arch. Virol. Suppl. 11, 135-140). The EBOV-mouse-adapted strain was generated by serial passage in progressively older mice (Bray et al., 1999, J. Infect. Dis. 179, Suppl 1, S248-258). EBOV was propagated and viral titers assessed by standard plaque assay in Vero E6 cells (Jahrling et al., 1996, supra; Bray et al., 1999, supra). Inactivated EBOV Zaire 1995 preparations were purified from cell-free supernatants on continuous sucrose gradients and irradiated with 1×10⁷ rads, as previously described (Hevey et al., 1997, Virology 239, 206-216). All experiments with EBOV were performed under maximum containment in a biosafety level (BSL)-4 laboratory at the United States Army Medical Research Institute of Infectious Diseases.

Mice. Female or male BALB/c, C57Bl/6, IFN-γ deficient (C57Bl/6 background), and perforin-deficient (BALB/c background) mice were obtained from National Cancer Institute (Frederick, Md.). NK cell-deficient mice were generated and bred at Washington University (St. Louis, Mo.) (Kim et al., 2000, Proc. Natl. Acad. Sci. USA 97, 2731-2736). NK cells were depleted from C57Bl/6 mice by intraperitoneal injection of 50 μl of anti-asialoGM antibodies (Wako Chemicals USA, Inc., Richmond, Va.) every other day from −5 to +5 days post challenge. Control mice were treated in the same manner using rabbit Ig (Sigma, St. Louis, Mo.). Mice (6-12 weeks old) were divided randomly into experimental groups, housed in microisolator cages, and provided food and water ad libitum. Research was conducted in compliance with the Animal Welfare Act and other federal statutes and regulations relating to animals and experiments involving animals and adhered to principles stated in the Guide for the Care and Use of Laboratory Animals, National Research Council, 1996. The facility where this research was conducted is fully accredited by the Association for Assessment and Accreditation of Laboratory Animal Care International.

VLP preparation. To generate VLPs, 293T cells were co-transfected with pWRG vectors encoding for EBOV VP40 and GP (VLP) or EBOV VP40 alone (VLP_{VP40}) using Lipofectamine 2000 (Invitrogen, Carlsbad, Calif.). To purify the VLPs, the cell-free supernatants were harvested after 2-3 days and pelleted at 9,500×g for 4 hours. These crude preparations were then separated on a 20-60% continuous sucrose gradient by ultracentrifugation overnight. The gradient fractions were concentrated by a second centrifugation, resuspended in endotoxin-free phosphate-buffered saline (PBS), and the fractions containing the VLPs were determined using western blots and electron microscopy. As a control, cell-free supernatants from 293T cells transfected with an empty pWRG vector were purified in an exact manner as the VLP preparations. Only a very small amount of cell-free supernatants from mock-transfected cells could be generated and experiments with these sucrose-purified supernatants resulted in similar outcome as medium alone. Therefore, the sucrose-purified cell-free supernatants were only used in select experiments. The amount of inactivated EBOV and VLP in each preparation was quantitated using a semi-quantitative western blot for VP40 along with a measurement of total protein concentration, obtained by disrupting the samples with NP40 detergent before use in a detergent-compatible protein assay (BioRad, Hercules, Calif.). The VLP preparations used in this study were <0.03 endotoxin units/mg, as determined by the Limulus amebocyte lysate test (Biowhittaker, Walkersville, Md.).

VLP injection and EBOV challenge of mice. For protection experiments, mice were injected intraperitoneally or intramuscularly with 25 μg of VLP, VLP_{VP40}, inactivated EBOV, or PBS alone 1, 2, or 3 days before challenge with mouse-adapted Ebola virus. Mice were challenged by intraperitoneal injection. As noted, mice were injected with 10 or 100 plaque forming units (pfu) of mouse-adapted EBOV (>300 or >3,000 LD₅₀, respectively) (Bray et al., 1999, supra). After challenge, mice were observed at least twice daily for illness and death for at least 28 days and no changes were observed in the health of any mice in these studies between 14 and 28 days post infection.

Flow cytometry. The spleen or mediastinal lymph nodes were collected from individual mice and placed in RPMI-1640 medium containing 10% fetal bovine serum (FBS), 2 mM L-glutamine, 1 mM HEPES, and 0.1 mM nonessential amino acids (referred to as complete RPMI). Single cell suspensions of lymphocytes were produced from each sample, the red blood cells were lysed with ACK lysis buffer, and the phenotypic expression of cells was examined by flow cytometry with NK1.1-FITC (BD Biosciences, San Jose, Calif.). Intracellular IFN-γ in NK cells was detected after fixation and permeabilization using Cytofix/Cytoperm™ (BD Biosciences), staining with PE-labeled IFN-γ, and analysis by flow cytometry, as described above. The percent of positive events were determined after collecting 50,000 events, gated based on forward and side scatter for viable lymphocytes, per sample using CellQuest software on a Becton Dickinson FACCalibur® (BD Biosciences, San Jose, Calif.).

Enrichment and depletion of NK cells. NK cells were isolated from the livers of mice following a hydrodynamic shearing method, which was used to increase the numbers of NK cells obtained from each mouse, unless noted (Liu et al., 1999, Gene Ther. 6, 1258-1266; He et al., 2000, Hum. Gene Ther. 11, 547-554). Briefly, mice received a hydrodynamic shear, or rapid tail vein injection, with 5 μg of IL-2 plasmid in 1.6 ml of 0.9% normal saline. Three to 4 days after the injection, lymphocytes were isolated using a 40%/80% Percoll® step gradient from perfused livers of the IL-2-treated mice. The NK cell preparations were obtained by negative selection using biotinylated CD3, CD4, CD8, and CD19 antibodies (BD Biosciences, San Jose, Calif.) followed by streptavidin-MicroBeads (Miltenyi Biotec Inc., Auburn, Calif.). The NK preparations were routinely 85-95% pure based on flow cytometry analysis for cell surface expression of NK1.1, both before and after overnight stimulation. The NK cell-enriched preparations contained 3-10% eosinophils, based on forward and side scatter and CD11b expression, 1-3% B220⁺MHC class II⁺ dendritic and B cells, and 1-2% CD5⁺ T cells, and did not contain CD3⁺ NK T cells (unpublished observations). To deplete the NK cells from the NK cell enriched preparations, the cells underwent a second negative selection using biotinylated NK1.1 antibodies (BD Biosciences, San Jose Calif.) and streptavidin-magnetic beads (yielded over 90% NK cell depletion).

Cell stimulations and blocking studies. NK cells (1×10⁶ cells/ml of complete RPMI) were stimulated for 2-72 hours with 100 iU/ml of murine IL-2 (PeproTech, Inc., Rocky Hill, N.J.), 10 μg/ml polyI:C, or 0.1-50 ug of inactivated EBOV, VLP, or sucrose-purified cell-free supernatants from mock-transfected cells. To assess the role of LPS contamination on NK cell cytokine secretion, 50 μg/ml of VLP or 10 ng/ml of LPS was incubated for 1 hour with 100 μg/ml of polymyxin B at room temperature (Jacobs and Morrison, 1977, J. Immunol. 118, 21-27) or boiled for 1 hour before their addition to NK cell preparations. In the blocking experiments, 10 μg of VLP was incubated with either a pool of three monoclonal antibodies (mAb) against EBOV GP (10 μg each) (Wilson et al., 2000, 1664-1666), 30 μg of an anti-EBOV VP40 mAb, 30 μl of mouse sera from mice vaccinated with either a replication-deficient Venezuelan equine encephalitis particle vaccine (VRP) expressing Ebola VP40 or Lassa N [a kind gift of M. K. Hart, (Wilson et al., 2001, Virology 286, 384-390)], or 30 μg of anti-human CD2 antibody (BD Biosciences). Percent inhibition of IFN-γ secretion was calculated as follows: [IFN-γ secretion with test antibody/IFN-γ secretion with control antibody (hCD2)]×100%.

Cytotoxicity assay. A standard 4-hour ⁵¹Cr assay was used to assess the cytotoxic activity of the stimulated NK cells (Yokoyama and Scalzo, 2002, Microbes Infect. 4, 1513-1521). Varying numbers of stimulated NK cells were added to 5,000 ⁵¹Cr-labeled YAC-1 target cells for 4 hours. The amount of ⁵¹Cr released into the supernatants of each sample was determined and the specific lysis was assessed by: [(sample cpm−spontaneous release)/(total release−spontaneous release)]×100%.

Cytokine detection. Concentrations of IFN-γ and TNF-α present in culture supernatants were measured by cytometric bead array (BD Biosciences, San Jose, Calif.) per the manufacturer's directions. The concentration of IFN-γ, MIP-1α, and TNF-α present in the EBOV-treated NK cell supernatants was tested by ELISA (R&D Systems, Minneapolis, Minn.) under BSL-4 containment.

NK cell transfers. After overnight stimulation, NK cells were washed twice and enumerated. Five million viable NK cells were resuspended in PBS and injected intraperitoneally into naïve mice. The recipient mice were challenged 6 hours later with EBOV and illness and survival were scored for 28 days.

Statistical analysis. A paired student's t test was used to directly compare treated and mock-treated samples. The proportion of treated and control animals surviving was compared by one-tailed Fisher exact tests within experiments. For survival experiments with more than one treatment group, adjustments for multiple comparisons were made by stepdown Bonferroni correction. Analyses were conducted using SAS Version 8.2 (SAS Institute Inc., SAS OnlineDoc, Version 8, Cary, N.C. 2000). A P value of 50.05 was considered significant.

### Example 1

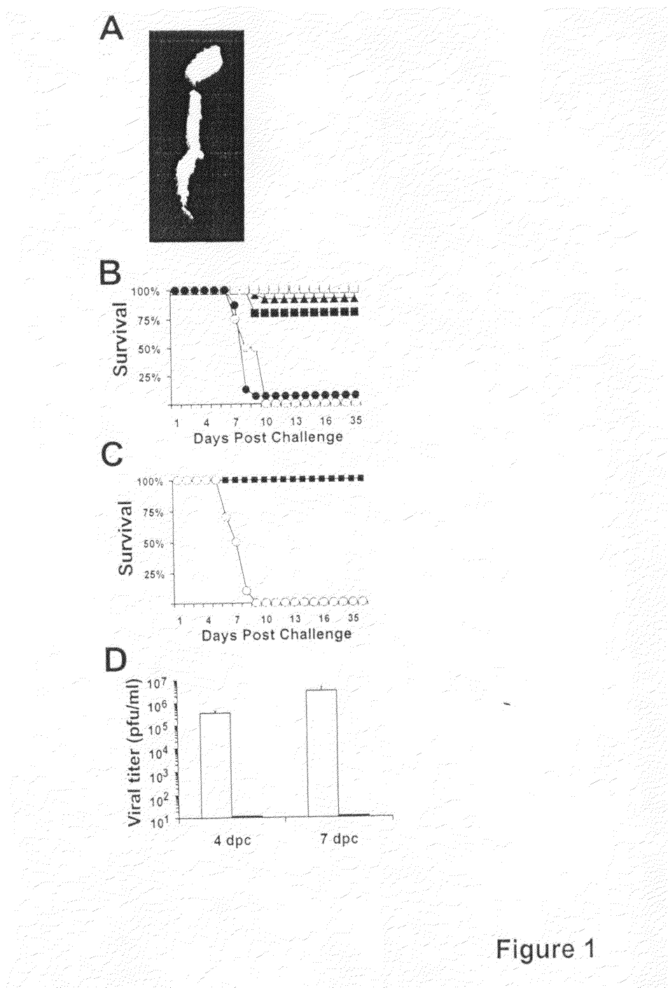

VLPs rapidly induce protection from lethal EBOV infection. Morphologically, the VLPs are almost indistinguishable from inactivated EBOV by electron microscopy (Warfield et al., 2003, supra; Bavari et al., 2002, J. Exp. Med. 195, 593-602) or by atomic force microscopy (FIG. 1A and (Feldmann et al., 2003, Nat. Rev. Immunol. 3, 677-685). The VLPs induced potent innate immune responses, as mice injected intraperitoneally once with VLPs, 1-3 days before challenge with more than 3,000 LD₅₀ of EBOV (Bray et al., 1999, supra) were 80-100% protected from death (FIG. 1B). However, mice injected 3 days before challenge with either irradiated, inactivated EBOV or the sucrose-purified supernatants from mock-transfected cells succumbed to EBOV challenge (FIG. 1B). Irradiating the VLPs had no effect on the outcome of these experiments (unpublished observations), suggesting that the failure of the inactivated EBOV to protect mice from EBOV infection was not simply due to the irradiation. Intramuscular injection of VLPs also induced high levels of protection against EBOV challenge (FIG. 1C), indicating the route of VLP administration was not linked to protection from EBOV lethality. Circulating Ebola virus was undetectable at 4 or 7 days after EBOV challenge in VLP-treated mice, while control mice exhibited high circulating viral titers following EBOV infection (FIG. 1D). Protection elicited within 1-3 days of VLP injection suggested that VLPs activated innate immune responses. Therefore, this approach gave us a vital tool to investigate early protective cellular responses to EBOV.

### Example 2

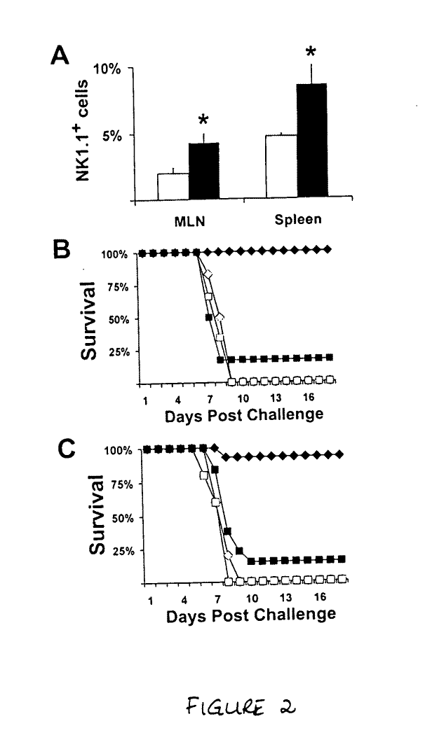

Innate protection against EBOV requires NK cells. Although many different factors may have contributed to VLP-induced innate protection, we narrowed our search to the role of NK cells. Marked increases in NK cell activity occur early in microbial invasions and results in the recruitment of NK cells to the site of infection (Yokoyama and Scalzo, 2002, supra). VLPs recruited almost twice the number of NK cells in both the mediastinal lymph node and spleen compared to animals receiving PBS alone (FIG. 2A), suggesting VLP administration induces NK cell proliferation and/or trafficking in lymphoid tissues. To directly examine the role of NK cells in EBOV infections, NK cell-deficient mice (Kim et al., 2000, supra) were administered VLPs 3 days prior to lethal EBOV challenge. VLP-pretreatment of mice lacking functional NK cells did not protect from EBOV infection (1/6, FIG. 2B), unlike VLP-injected wild-type C57Bl/6 mice (6/6, P=0.0076). Further, mice depleted of NK cells using anti-asialoGM1 antibodies were not protected by VLP treatment (2/13 survivors, FIG. 2C), unlike VLP-treated C57Bl/6 mice (14/15 survivors, P=0.0001). While anti-asialoGM1 antibodies can deplete both NK cells and cells of a monocytic lineage, together these data directly implicated NK cells in the rapid protection mediated by VLPs.

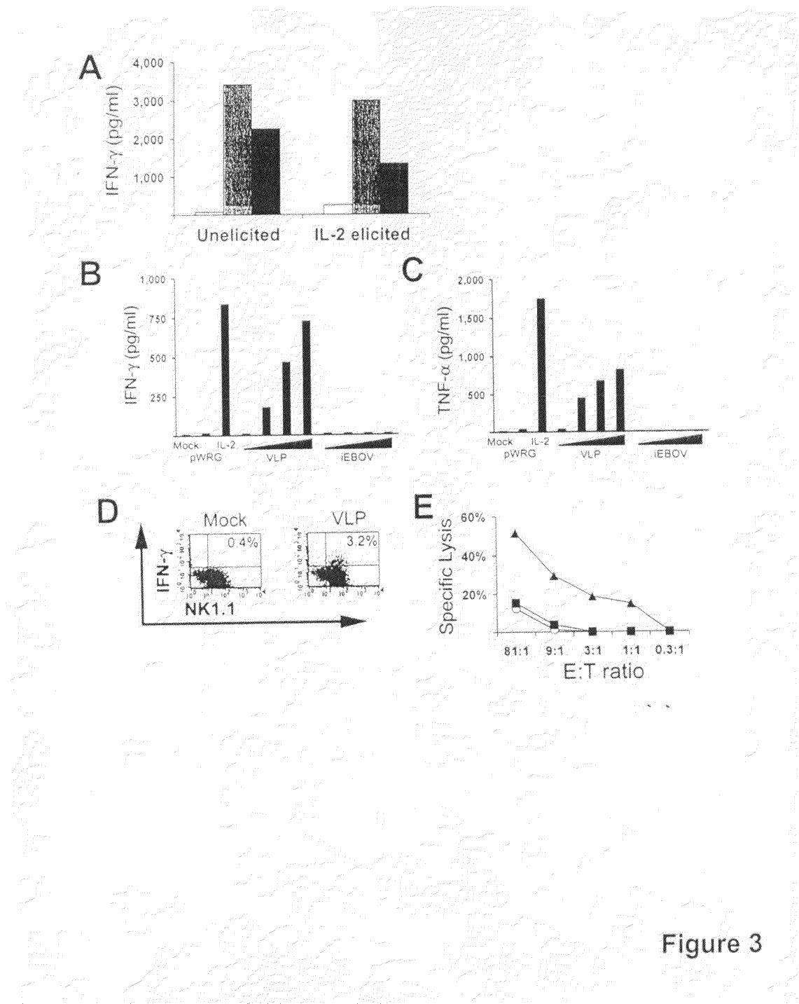

Since NK cells were required for protection against EBOV infection, we examined whether VLPs induced the functional activation of NK cells in vitro. In order to enhance the number of NK cells and to obtain highly enriched preparations of NK cells, we employed a cDNA hydrodynamic shearing method (Liu et al., 1999, supra; He et al., 2000, supra). Following the rapid tail vein injection of IL-2 plasmid, a substantial increase was observed in the number of NK cells in the liver (unpublished observations). To determine the effect of this procedure on NK cells, we obtained NK cells from livers of untreated or sheared C57Bl/6 mice and found no differences in cytokine profiles when these cells were stimulated with IL-2, VLPs, or sucrose-purified cell-free supernatants from mock-transfected cells (FIG. 3A). The VLPs, but not inactivated EBOV, induced IFN-γ and TNF-α secretion from NK cells (FIG. 3B-C). NK cells activated with VLPs also secreted IL-4, IL-5, IL-6, IL-13, and MIP-1α, but not detectable IL-2 and IFN-α (unpublished observations). We performed intracellular staining for IFN-γ and surface staining for NK1.1 to confirm that the NK cells were the main producers of IFN-γ. There was a considerable increase in the number of IFN-γ⁺ and NK1.1⁺ cells after VLP stimulation, as compared to NK cells incubated overnight in media alone (FIG. 3D). These IFN-γ⁺, NK1.1⁺ cells did not express CD3 (unpublished observations) and thus NK, not NK T, cells were specifically responsible for IFN-γ secretion. To show that this stimulation was the result of VLP preparations and not endotoxin contamination, VLPs or lipopolysaccharide were boiled or treated with polymyxin B, a compound that binds and neutralizes the biological activity of lipopolysaccharide (Jacobs and Morrison, 1977, supra), and then the preparations were added to purified NK cells. Denaturation of VLPs by boiling, but not polymyxin B treatment, abrogated the NK cytokine responses; the opposite was true for lipopolysaccharide (unpublished observations). NK cells stimulated with VLPs for 18 hours, but not 2 hours, specifically killed susceptible YAC-1 target cells (FIG. 3E). These results show that Ebola VLPs induced strong NK cytotoxic activity, as well as cytokine and chemokine secretion.

### Example 3

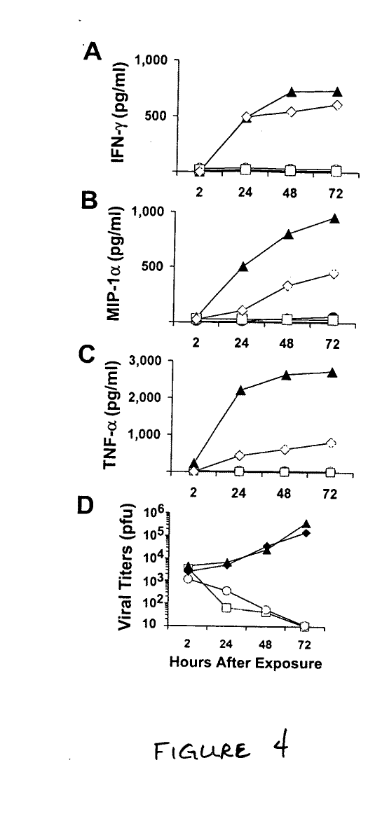

NK cell responses to Ebola virus. Ebola VLPs are morphologically and antigenically similar to live EBOV [FIG. 1A and Warfield et al., 2003, supra; Bavari et al., 2002, supra; Swenson et al., 2004, FEMS Immunol. Med. Microbiol. 40, 27-31]. However, unlike VLPs, inactivated EBOV did not induce innate protection from EBOV infection or stimulate NK cell responses in vitro (FIG. 1B). Therefore, we set out to determine if murine NK cells possessed the ability to respond to live EBOV. Unlike exposure to IL-2 or VLPs, live EBOV did not induce secretion of IFN-γ, MIP-1α, or TNF-α from NK cells (FIG. 4A-C).

Several viruses, including human cytolomegalovirus, HIV, and Epstein-Barr virus replicate efficiently in NK cells (Rice et al., 1984, Proc. Natl. Acad. Sci. USA 81, 6134-6138; Chehimi et al., 1991, J. Virol. 65, 1812-1822; Kanegane et al., 2002, Crit. Rev. Oncol. Hematol. 44, 239-249; Valentin and Pavlakis, 2003, Anticancer Res. 23, 2071-2075). To determine whether the lack of NK cell responses to EBOV were caused by EBOV infection of the NK cells, we determined the viral titers in supernatants of murine NK cells exposed to EBOV (moi=1, Zaire 95 or mouse-adapted). Ebola virus did not replicate in NK cells; in fact, the amount of live virus in the supernatants dropped during the 72 hours after exposure to virus (FIG. 4D). The inability of EBOV to replicate in NK cells was not due to death of the NK cells, as mock-infected and EBOV-infected NK cells had nearly the same viability after 3 days in culture (unpublished data). As expected, both viruses grew quickly to high titers in permissive VeroE6 cells [FIG. 4D and Jahrling et al., 1996, supra; Bray et al., 1999, supra]. Neither wild-type EBOV-Zaire nor the mouse-adapted strain of EBOV stimulated cytokine secretion in NK cells nor replicated efficiently in murine NK cells, indicating the mouse-adapted EBOV does not differ drastically from the wild-type EBOV-Zaire in regards to the effects on NK cells (FIG. 4A-C).

### Example 4

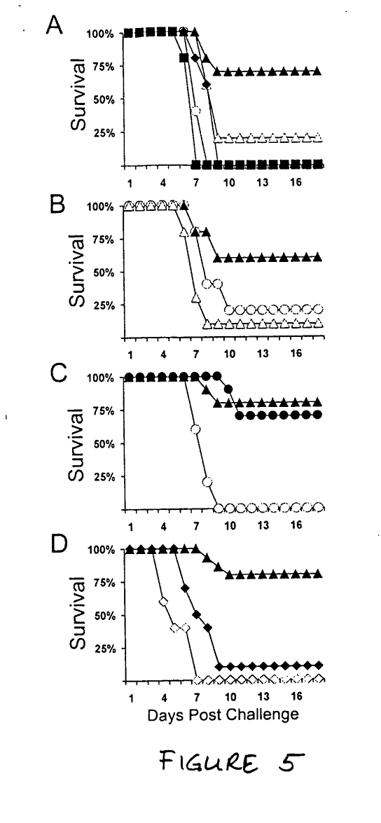

NK-cell mediated protection against EBOV is perforin-dependent. Collectively, our observations prompted us to determine whether these functional responses of the VLP-exposed NK cells could reconstitute the short-term protection from EBOV observed in mice injected with VLPs. To do this, VLP-treated NK cells were transferred to naïve mice, and then the mice were challenged with EBOV. Animals treated with NK cells stimulated with a 10 μg dose of VLPs showed high survival rates (14/20, survivors/total) and even those mice that were treated with NK cells that had been stimulated with a low dose of VLPs developed enhanced protection against EBOV challenge (FIG. 5A). In contrast, none of the mice receiving NK cells treated with either 50 μg/ml of inactivated EBOV, 10 μg/ml polyI:C, or media alone survived (FIG. 5A). Mice that failed to survive, but received VLP-stimulated NK cells, survived longer after EBOV infection than mice administered unstimulated NK cells (FIG. 5A). To confirm that the NK cells, and not another cell type, were required for protection from EBOV infection, NK1.1⁺ cells were depleted (>90% removed) from the standard NK cell preparation and the remaining cells in the preparation were transferred following overnight incubation with VLPs. The preparation containing NK cells, but not the NK1.1⁺ cell-depleted preparation, protected animals from lethal EBOV infection (FIG. 5B). VLP-stimulated NK cells from IFN-γ deficient mice resulted in a high level of survival (FIG. 5C), similar to NK cells from wild-type mice. In contrast, VLP-stimulated NK cells isolated from perforin-deficient mice did not elicit protection from EBOV infection (FIG. 5D). Thus, although IFN-γ conventionally plays a major role in innate viral infection, this cytokine was apparently not involved in innate protection against EBOV; however, the protection was tightly connected to perforin-dependent cytotoxic activity of the NK cells treated with VLPs.

### Example 5

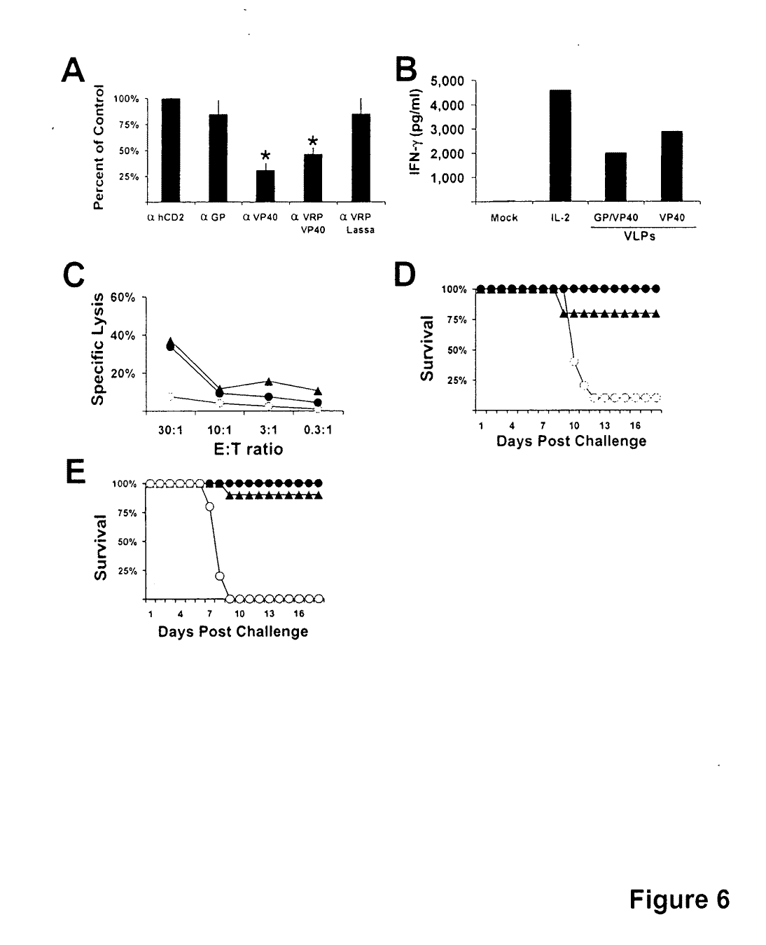

Ebola VP40 is sufficient to induce NK responses. The Ebola VLPs are enveloped particles comprised of the glycoprotein GP and the matrix VP40, which bud from cellular lipid rafts (Bavari et al., 2002, supra). We sought to determine whether one of these viral components of the VLPs was responsible for the induction of NK responses. A single mAb against VP40, but not a pool of three mAbs against GP or irrelevant antibody (anti-human CD2), was able to block IFN-γ secretion by the VLP-stimulated NK cells (FIG. 6A). Sera from mice vaccinated with VRP encoding Ebola VP40 blocked IFN-γ secretion induced by the VLPs, while control sera from mice vaccinated with a VRP encoding the Lassa virus glycoprotein had no effect (FIG. 6A).

To further examine the role of VP40, we took advantage of the fact that expression of EBOV VP40 alone in mammalian cells also results in generation of VLPs (VLP_{VP40}), although with lower efficiency than with expression of both GP and VP40 (35, 36). NK cells stimulated overnight with VLP_{VP40} secreted cytokines, including IFN-γ (FIG. 6B). Additionally, VLP_{VP40}-treated NK cells displayed cytotoxic activity against susceptible targets, similar to NK cells treated with VLPs (FIG. 6C). When NK cells were stimulated overnight with VLP_{VP40} and transferred to naïve mice, they fully protected mice from lethal challenge with EBOV infection (FIG. 6D). Additionally, mice administered VLP_{VP40} three days prior to infection with mouse-adapted EBOV were completely protected from this lethal challenge (FIG. 6E). These data suggest that the main viral protein involved in the innate immune responses to VLPs, including the NK-mediated protective effect, is the matrix protein VP40.

Discussion

We have established a model system to examine Ebola virus pathogenesis using hollow, genome-free VLPs. The VLPs swiftly induced effective protective immune responses in mice. This innate protection was dependent on NK cells, since NK cell-deficient and NK cell-depleted mice were not protected from EBOV by the VLPs. NK cells exposed to VLPs secreted pro-inflammatory cytokines and chemokines and killed susceptible target cells. Further, the transfer of VLP-activated NK cells was sufficient to elicit substantial protection against lethal filovirus infection in mice. The mechanism of innate protection against EBOV was not dependent on IFN-γ, but perforin was required. The protective effect of the VLP-induced NK cell activity was due mainly to the viral matrix protein VP40.

Functional changes in NK cells were not detected following exposure to live or inactivated EBOV. NK cells did not secrete cytokines, including IFN-γ, TNF-α, or MIP-1α, in response to EBOV. Similarly, our in vivo studies have suggested that EBOV infection of mice or monkeys does not to activate significant NK cell responses (unpublished observations). EBOV may actively interfere with or avoid innate immune responses, including NK responses (Mahanty et al., 2003, supra; Bosio et al., 2003, supra). EBOV GP has been proposed to modulate host adaptive immune responses (Feldmann et al., 1999, Arch. Viol. Suppl. 15, 159-169). However, GP does not interfere with early innate immune responses, specifically NK cell responses, in the context of VLPs, since protective immune responses are elicited by both VLPs and VLP_{VP40}. EBOV VP35 is the other known immune modulator and has been identified as an IFN antagonist. In EBOV-infected cells, VP35 blocks phosphorylation and dimerization of interferon regulatory factor 3, effectively preventing transcription of key antiviral genes (Bosio et al., 2001, supra; Basler et al., 2000, Proc. Natl. Acad. Sci. USA 97, 12289-12294; Basler et al., 2003, J. Virol. 77, 7945-7956). While EBOV was not able to replicate efficiently in murine NK cells, it is possible that the virus was able to bind to, or enter, these cells and interfere with their response to the viral antigens through VP35 or other viral proteins. Although the mechanisms are unclear at this time, the virulence of EBOV may depend on its ability to evade or down-regulate the innate immune cell responses to viral infections, especially early responders such as NK cells. In fact, there is a specific loss of NK cells and a decrease in NK cell function following EBOV infection of primates (Ignatiev et al., 2000, Immunol. Lett. 71, 131-140; Geisbert et al., 2003, Am. J. Pathol. 163, 2347-2370; and unpublished observations]. Taken together with our current findings, these data indicate a role for NK cells in the pathogenesis of EBOV.

Viral proteins are capable of directly inducing NK cell responses (Yokoyama and Scalzo, 2002, supra). Filovirus glycoproteins (GPs) represented the most likely candidates for interacting with NK cells directly, as the two other viral proteins known to directly induce NK cell responses are also GPs. The murine activating receptor Ly49H directly recognizes a MCMV-encoded glycoprotein m157, which is a MHC-like molecule (Yokoyama and Scalzo, 2002, supra; Arase et al., 2002, Science 296, 1323-1326). The NKp44 and NKp46 receptors on human NK cells interact with the influenza virus glycoprotein hemagglutinin via sialic acid side chains, leading to the NK cell-mediated lysis of influenza virus-infected cells (Mandelboim et al., 2001, Nature 409, 1055-1060; Arnon et al., 2001, Eur. J. Immunol. 31, 2680-2689). In contrast, we found that the viral matrix protein VP40, and not EBOV GP, is critical and sufficient for the induction of innate, and specifically NK cell, responses to EBOV. Previously, EBOV GP has been presumed to be the only viral protein exposed on the surface of the virion. However, it is possible that VP40 is partially exposed on the virus surface. A recent report indicates that mAb against the Marburg virus VP40 protein are capable of inducing complement-mediated lysis of infected cells (Razumov et al., 1998, Vopr. Virusol. 43, 274-279). Crystallographic data show that VP40 can form octamers with a central pore, reminiscent of pore-forming toxins that insert into the plasma membrane (Gomis-Ruth et al., 2003, Structure (Carob) 11, 423-433). Furthermore, VP40 possesses integral membrane association characteristics and oligomerizes in the rafts of host cell membranes prior to driving virus particle formation (Panchal et al., 2003, Proc. Natl. Acad. Sci. USA 100, 15936-15941; Jasenosky et al., 2001, J. Viol. 75, 5205-5214). Therefore, it is possible that VP40 is partially exposed on the surface of VLPs, and that this might be important for the stimulatory effect of these particles on innate immune cells. We propose that recognition of VP40 may be critical for alerting early, innate immune responses, while the immune responses to GP plays a more important role in the subsequent generation of protective adaptive immune responses.

In contrast to NK cells from wild-type C57Bl/6 mice, VLP-stimulated NK cells isolated from perforin-deficient mice failed to protect naïve mice from lethal EBOV infection. Perforin-mediated NK cytotoxicity has a well-established role in tumor surveillance (van den Broek and Hengartner, 2000, Exp. Physiol. 85, 681-685) and has a recognized, but less appreciated, role in viral infections (Ghiasi et al., 1999, Virus Res. 65, 97-101; Tay and Welsh, 1997, J. Virol. 71, 267-275). Our data are in line with previous findings where control of HSV-1 infection in the eye and MCMV infection in the spleen of adult mice is mediated via a perforin-dependent mechanism (Ghiasi et al., 1999, supra; Tay and Welsh, 1997, supra; Tay et al., 1999, J. Immunol. 162, 718-726). NK cell cytotoxic activity can be directly activated by receptor-ligand interactions or induced by exposure to cytokines including IFN-α/β, TNF-α, or IL-12 (Biron et al., 1999, supra). However, it is not yet clear whether the cytotoxic activity of VLP-stimulated NK cells is a direct effect, or the result of secondary stimulation mediated by cytokine production. The production of cytokines such as IFN-α/β, IFN-γ, and TNF-α by NK cells is important for both the direct and indirect antiviral activity of NK cells (Biron et al., 1999, supra). Treating NK cells with VLPs induced considerable secretion of TNF-α, IFN-γ, and other pro-inflammatory cytokines in vitro. The cytokine responses to viral antigens was not due to priming by IL-2 pre-treatment of the mice, as NK cells from the livers of untreated C57Bl/6 mice secreted cytokines in a similar pattern to that secreted by IL-2-treated mice after exposure to VLPs (unpublished observations). However, IFN-γ does not appear to be essential for the protective action of VLPs, as cells from IFN-γ knockout mice were fully capable of conveying protection.

NK cells are activated through a variety of ligand-receptor interactions (Yokoyama and Scalzo 2002, supra). NK cells stimulated with VLPs did not induce changes in the levels of cell surface NK activating or inhibitory receptors, and we were also unable to identify a specific population of NK cells associated with the IFN-γ secretion (unpublished observations). Further, VLP-stimulated NK cells from BALB/c mice secreted cytokines in a similar manner to C57Bl/6 mice and protected against EBOV challenge when transferred to naïve mice (unpublished observations and FIG. 5D). Therefore, VLP stimulation of NK cells is not restricted to a single mouse strain, and it is not related to the expression of Ly49H activating receptor (Brown et al., 2001, Science 292, 934-937). It is possible that NK cell activation by VLPs may not be receptor-mediated, but may be mediated purely by cytokines or other unidentified mechanisms.

PolyI:C-treatment of NK cells significantly increases protection against HSV-1 infection, when compared to protection provided by untreated cells (Rager-Zisman et al., 1987, supra). In contrast, we found that polyI:C-treatment of NK cells prior to transfer did not confer protection from EBOV infection (FIG. 5A), indicating non-specific stimulation of NK cells is not sufficient for protection. In support of these findings, CpG-treatment of the NK cell preparations prior to transfer did not protect naïve mice from EBOV challenge (unpublished observations), further suggesting that activation of antigen-presenting cells in NK cell preparations could not account for the observed protection. Therefore, the protection provided by VLP-treated NK cells appears to be mediated by VLP-specific responses, although we do not understand the mechanisms of action at this time.

We were concerned that NK T cells contributed to the biological responses of our VLP-exposed cellular preparations. However, the NK cell-enriched preparations did not contain CD3⁺ NK T cells, but were contaminated with eosinophils (<10%), B220⁺MHC class II⁺ cells (<3%) that could be macrophages, dendritic or B cells, and a small number of CD5⁺ T cells (<2%). Depletion of NK1.1⁺ cells from the cell preparations prior to transfer abrogated innate protection from EBOV, suggesting that contaminating antigen-presenting cells, eosinophils, or other lymphocytes were not required for innate responses to EBOV. Nonetheless, it may be that VLPs are taken up by DCs or macrophages, which in turn activate the NK cells or that VLPs are rapidly processed and presented by the antigen-presenting cells to B or T cells. In contrast, exposure to inactivated EBOV does not activate or mature murine DCs (18) and thus, likely does not efficiently prime secondary lymphocyte responses. We have previously shown that both B and T lymphocytes are activated transiently 2-3 days post challenge in the lymph nodes of VLP-vaccinated mice (18). Changes in early T cell activation markers, including CD25, CD43, and CD69, are not detectable until at least 48 hours post injection in lymph nodes, spleen, or peritoneal cavity and thus, do not exactly correlate with the rapid protection observed in our current study within one day post injection [unpublished data and Warfield et al., 2003, supra]. In this report, we have shown a critical involvement of NK cells in innate protection against EBOV infection; however, at this time, we cannot rule out the contribution of other cell types, including dendritic, B, and T cells.

The innate immune system provides early surveillance and control of viral infections. In this report, we show that the innate immune response, specifically NK cells, can mediate rapid and complete protection against lethal EBOV infection. These observations represent a key advance in understanding the requirements for protective immunity against EBOV infection. The identification of NK cells as critical mediators of early protection against EBOV infection are an important step forward in the identification of prophylactic and therapeutic interventions against filovirus and other incapacitating acute viral infections as well as providing therapeutic agents which bolster the innate immune response, including activation of NK cells.

What is claimed is:

1. A method for activating an immune system of an animal which comprises administering filovirus virus like particles, VLPs, in an amount effective to activate an immune response in the animal.

2. The method of claim 1 wherein said filovirus is chosen from the group consisting of Ebola and Marburg.

3. The method of claim 1 wherein said VLPs activate innate immunity.

4. The method of claim 1 wherein said VLPs activate natural killer, NK, cells.

5. A composition for activating an immune system comprising filovirus VLPs.

6. The composition according to claim 5 wherein said VLPs activate NK cells.

7. A composition for enhancing an immune response to an antigen in an animal comprising filovirus VLPs and said antigen.

8. The composition of claim 7 wherein said antigen is bound to the VLPs.

9. The composition of claim 8 wherein said antigen is mixed with the VLPs.

10. The composition of claim 7 wherein said filovirus is chosen from the group consisting of Ebola and Marburg.

11. The composition of claim 7 wherein said VLP comprises VP40 chosen from Ebola VP40 and Marburg VP40.

12. The composition of claim 11 wherein said VLP further comprises GP from one or more filovirus chosen from the group consisting of Ebola Zaire 1976, Ebola Zaire 1995, Ebola Reston, Ebola Sudan, Ebola Ivory Coast, Marburg Musoke, Marburg Ravn, Marburg Ozolin, Marburg Popp, Marburg Rataczak, and Marburg Voege.

13. The composition of claim 13 further comprising other filovirus proteins chosen from the group consisting of NP, VP24, VP30, and VP35.

14. The composition of claim 7 where said antigen is a recombinant antigen.

15. The composition of claim 7 wherein said antigen is isolated from a natural source.

16. The composition of claim 15 wherein said natural source is selected from the group consisting of: pollen extract, dust extract, dust mite extract, fungal extract, mammalian epidermal extract, feather extract, insect extract, food extract, hair extract, saliva extract, and serum extract.

17. The composition of claim 7 wherein said antigen is derived from the group consisting of viruses, bacteria, parasites, prions, tumors, self-molecules, non-peptide hapten molecules, allergens, and hormones.

18. The composition of claim 7 wherein said antigen is a tumor antigen.

19. A vaccine comprising an immunologically effective amount of the composition of claim 7.

20. The vaccine of claim 7 further comprising an adjuvant.

21. A method for immunizing or treating an animal comprising administering to said animal an immunologically effective amount of the vaccine of claim 19.

22. The method of claim 21 wherein said composition is introduced into said animal subcutaneously, intramuscularly, intravenously, intranasally or directly into the lymph node.

23. The method of claim 21 wherein said animal is a mammal, preferably a human.

22. Use of the composition of claim 7 in the manufacture of a pharmaceutical for the treatment of a disorder or disease selected from the group consisting of: allergies, tumors, chronic diseases an chronic viral diseases.
